(19) United States
(12) Patent Application Publication
(10) Pub. No.: US 2007/0293831 A1
(43) Pub. Date: Dec. 20, 2007

(54) Medicament Application Device

(75) Inventor: Kenneth W. Kwochka, St. Joseph, MO (US)

Correspondence Address: RATNERPRESTIA, P O BOX 980, VALLEY FORGE, PA, 19482-0980, US

(73) Assignee: IVX ANIMAL HEALTH, INC., ST. JOSEPH, MO (US)

(21) Appl. No.: 10/582,428

(22) PCT Filed: Dec. 10, 2004
(86) PCT No.: PCT/US04/41463

§ 371 Date: Aug. 2, 2007

Related U.S. Application Data
(60) Provisional application No. 60/529,008, filed on Dec. 11, 2003.
(60) Provisional application No. 60/577,396, filed on Jun. 4, 2004.

(51) Int. Cl. A61M 35/00

(52) U.S. Cl. 604/310

(57) ABSTRACT

A medicament application device for using with a medicament container includes a plate (18) defining at least one hole (26) therethrough, a connector (20) extending from a bottom side of the plate to a proximal end, and a medicament applicator (22) detachably attached to a top side of the plate. The medicament applicator is made from a fluid permeable material. The proximal end of the connector is adapted for connecting to an opening of the medicament container. The connector defines an inner channel, which is in fluid communication with the opening of the container and the hole of the plate.

### FIELD OF THE INVENTION

This invention relates to medicament application devices, and more specifically, to medicament application devices for dispensing liquid medicament from a reservoir to the skin of a mammal for treating skin disorders.

### BACKGROUND OF THE DISCLOSURE

The delivery of a liquid medicament to the skin of a mammal, such as a human, a cat, a dog, or a horse, for treating a skin disorder or disease is desired in many circumstances. In the prior art, a user uses fingers or a portable sponge or pledget to apply medicament to the skin where the disorder occurs. This type of method is non-efficient in the application of the medicament to a large area of the skin, and may cause transmission of infectious diseases from the skin to the user's hand. Other devices used in the prior art, for example, a spray, fail to apply the medicament directly and evenly to the skin, with the effect that the medicament may not be distributed and/or absorbed effectively. Therefore, there is need for a new medicament application device that can effectively apply the liquid medicament to the skin of the mammal and can reduce the risk of transmission of a skin disease to other animals and to humans.

It is an object of the present invention to provide an improved medicament application device for treating a skin disorder or disease of a mammal.

It is a further object to provide an improved medicament application device that reduces the risk of transmission of the skin disease to other animals and to humans.

### SUMMARY OF THE INVENTION

The present invention provides a medicament application device to apply liquid medicament to the skin of a mammal for treating a skin disorder or disease.

According to one aspect of the present invention, the medicament application device includes a plate, a connector extending from a bottom side of the plate for connecting the device to a liquid medicament container, and a medicament applicator attached to a top side of the plate. The plate defines at least one hole through the plate, and the connector defines an inner channel, which is in fluid communication with the hole on the plate. In use, the connector is connected to an opening of the container, so that a continuous fluid passageway is provided from the liquid medicament-containing interior of the container, through the inner channel of the connector and the hole on the plate, to the medicament applicator. The hole, or holes, may have a cross-section of any desired shape, such as circular, rectangular, or elliptical. The holes may alternatively be “star-shaped”. The hole geometry may be effective to optimally permit delivery of medicament to a bottom side of the applicator, preferably, in a spatially uniform manner.

The container, if flexible, may be squeezed by a user to drive the liquid medicament to the applicator. Alternatively, by inverting the application device/container assembly, the liquid medicament may be gravity-driven to the applicator.

The medicament applicator is preferably made from a material that allows fluid to pass through, and at the same time, spread the fluid to a whole application surface of the applicator. Exemplary materials for the applicator include, but are not limited to, flexible porous materials such as, a sponge and cloth. The liquid medicament contained in the container can pass through the passageway to the medicament applicator, and through the applicator to the skin of the mammal. A user can move the device across the affected area of the skin, squeeze the applicator by pushing the device against the skin, and apply the liquid medicament to that skin area evenly.

The medicament applicator is preferably replaceable and disposable. In one preferred embodiment, the applicator includes a sponge and a cover which is preferably made from a mesh. Preferably, but not necessarily, the plate defines a central concave region at the top side of the plate for receiving a lower portion of the sponge. In use, the lower portion of the sponge is received in the central concave region of the plate, and the top of the sponge is covered by the mesh. The mesh includes holes near its periphery which are affixable to hooks extending from the bottom side of the plate, so that the sponge may be secured on the plate and is not movable during use.

In another preferred embodiment, the plate includes an array of resilient hooks, such as VELCRO™ hooks, extending from the top side of the plate, and the medicament applicator is a cloth or other material having a bottom surface adapted to engage the hooks. The cloth is detachably secured to the plate by attaching the bottom surface of the cloth to the hooks.

In a further preferred embodiment, the plate defines relatively small holes in a pattern and the applicator includes a relative base which supports a porous material, with the base including relatively small protrusions extending from the bottom side of the applicator with the same pattern. The small protrusions can be pushed to snap-fit into the associated relatively small holes defined on the plate, so that the applicator is secured on the plate.

The applicator can be removed from the plate by pulling the applicator in an opposite direction, or by pushing the protrusions out of the holes from the bottom side of the plate. The applicator then can be replaced.

In an alternative preferred embodiment of the present invention, the applicator is in the form of bristles, which extend from a base which preferably is removably attachable (for example, with holes and protrusions) to the top side of the plate for applying medicament to the skin of the mammal. The base defines multiple holes among the bristles. The holes are connected via holes in the plate to the inner channel of the connector, which is connected to the container, for supplying medicament to the bristles from the container. In use, the liquid medicament passes through the inner channel and the holes to the bristles, and the bristles guide the application of the liquid medicament to the skin of the mammal.

In another preferred embodiment, the medicament application device includes a connector for connecting to the medicament container, a plate connected to the connector at a back side, and an applicator attached to a front side of the plate. The connector of the medicament application device includes a plate portion and a stem portion extending from the back of the plate portion. The stem portion of the connector is adapted for connecting to the stem of the medicament container. The plate of the medicament application device includes walls extending upward from the back side of the plate and flanges extending inward from the top of the walls. The walls define an interior region with an opening at a back edge of the plate. A tab, which is preferably resilient, extends from the back edge of the plate, and a relatively small protrusion or flange extends upward from the tab. The device can be assembled by sliding the plate portion of the connector into the interior region from the opening over the relatively small flange. The plate portion then is locked in the interior region by the walls, the flanges, and the relatively small flange on the tab. The device also can be disassembled by pressing the tab downward and sliding the plate portion backward out of the interior region from the opening. The applicator is attached to the front side of the plate, preferably, by gluing or other means. The applicator is preferably made from a flexible porous material, for example, sponge.

The stem portion of the connector defines an inner channel therethrough, and the plate portion of the connector and the plate define holes in a pattern that can optimally permit delivery of medicament to a bottom side of the applicator, preferably, in a spatially uniform manner. The plate and the applicator can be released from the connector as described above, and can be replaced, thereby preventing transmission of skin diseases from one animal to another.

### BRIEF DESCRIPTION OF THE DRAWINGS

For a fuller understanding of the nature and the objects of the invention, reference should be made to the following detailed description and the accompanying drawings in which like reference numerals refer to like elements and in which:

FIG. 1 is a front view of a medicament application device in use with a medicament container according to one preferred embodiment of the present invention;

FIG. 2 is a side view of the medicament application device in FIG. 1 showing that the device is in use with a medicament container;

FIG. 3 shows an enlarged side view of the medicament application device in FIG. 1;

FIG. 4 shows an enlarged front view of the medicament application device in FIG. 1;

FIG. 5 shows an enlarged back view of a medicament applicator used with the medicament application device in FIG. 1;

FIG. 6 shows a perspective front view of the medicament application device in FIG. 1, showing that a medicament applicator is received in a central concave region in a plate;

5 FIG. 7 shows a perspective front view of the medicament application device in FIG. 1, showing that a medicament applicator is secured to the plate by a mesh netting;

FIG. 8 is a front view of a medicament application device in use with a medicament container according to another preferred embodiment of the present invention;

FIG. 9 shows an enlarged front view of the medicament application device in FIG. 8;

FIG. 10 shows a perspective side view of the medicament application device according to another preferred embodiment of the present invention;

FIG. 11 shows a front view of the medicament application device in FIG. 9;

FIG. 12 shows a perspective side view of the medicament application device according to another preferred embodiment of the present invention;

FIG. 13 shows a front view of the medicament application device in FIG. 12;

FIG. 14 shows a perspective front view of the medicament application device according to another preferred embodiment of the present invention;

FIG. 15 a perspective back view of the medicament application device in FIG. 14;

FIG. 16 shows a perspective front view of the medicament application device according to another preferred embodiment of the present invention;

FIG. 17 shows a perspective view of the medicament application device according to another preferred embodiment of the present invention;

FIG. 18 shows another perspective view of the medicament application device in FIG. 17, viewed from a different angle;

FIG. 19 is an exploded view of the medicament application device in FIG. 17;

FIG. 20 is a perspective view of a plate of the medicament application device in FIG. 17;

FIG. 21 is a back view of a connector of the medicament application device in FIG. 17;

FIG. 22 is a front-side view of the connector shown in FIG. 21;

FIGS. 23A and 23B show front views of the medicament application device in accordance with one preferred embodiment;

FIGS. 24A-24C show back views of the medicament application device in FIGS. 23A and 23B;

FIG. 25 shows a side view of the medicament application device connected with a medicament container, and

FIG. 26 shows a rinse kit according one preferred embodiment of the present invention.

### DESCRIPTION OF THE PREFERRED EMBODIMENTS

The invention is a medicament application device which dispenses fluid medicament from a reservoir to the skin of a mammal for treating skin disorders or for cleaning the skin of the mammal. Exemplary embodiments of the invention are shown in the figures and described in the following description.

Topical medicaments to be used with the Medicament Application Device include but are not limited to moisturizing bath rinses (e.g., oils, humectants, fatty acids) such as HyLyt® Bath Oil; non-steroidal and steroidal anti-inflammatories and/or anti-pruritics (e.g., anesthetics, antihistamines, glucocorticoids) such as Relief® Spray or CortiSpray®; anti-infectives (e.g., lime sulfur, chlorhexidine, miconazole, ketoconazole, enilconazole, mupirocin) such as Malaseb™ Rinse, Malaseb™ Spray, and LymDyp™; anti-parasitics (e.g., pyrethrins, pyrethroids, avermectins).

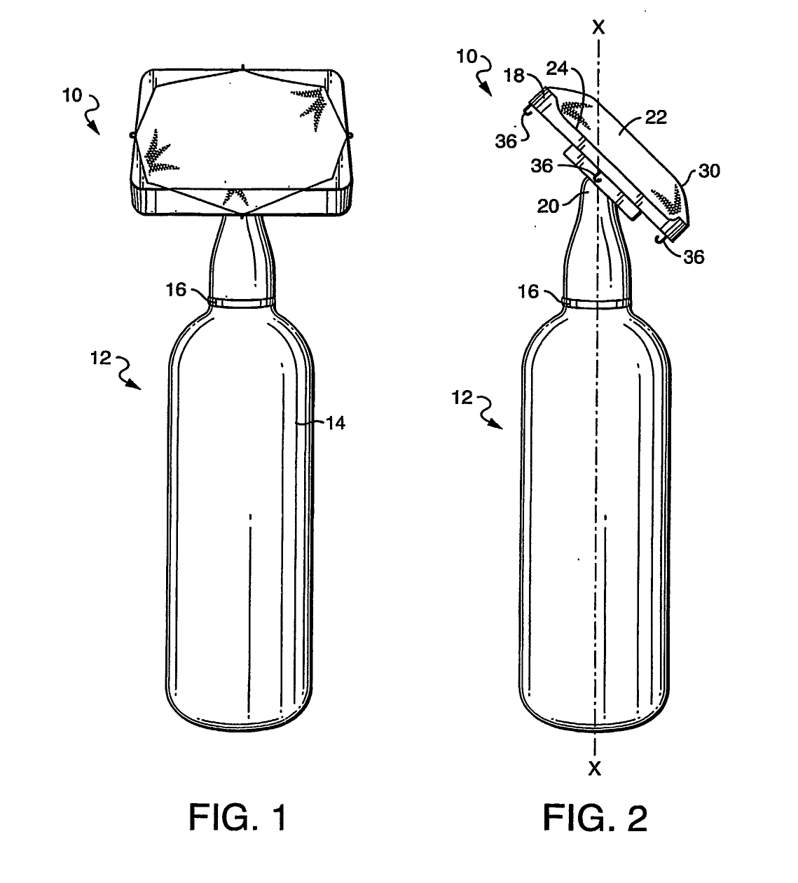

One exemplary medicament application device 10 embodying the present invention is illustrated in FIG. 1. As shown in FIG. 1, the medicament application device 10 is used with a medicament container or reservoir 12 which has a bottle shape body 14 and a stem 16 extending from the top of the body 14. The stem 16 defines an opening at the top of the stem for dispensing medicament from the bottle body 14 to the medicament application device 10, which is connected to the container 12. In one preferred embodiment, the stem 16 is threaded and the medicament application device 10 includes threads at the bottom end for engaging the threads of the stem 16. The medicament application device 10 also can be attached to the stem 16 by other means, for example, snap-fitting. It should be understood that the present invention can also be used with reservoirs or containers with other shapes. The container can be rigid or flexible. The container 12 contains rinse, preferably concentrated rinse, at an appropriate concentration of the medication, formulated for mammals, for example, dogs, cats, or horses.

FIG. 2 shows a side view of the device 10, which is used with the container 12. The medicament application device 10 includes a plate 18, a connector 20 extending from a bottom side of the plate 18 for connecting the device 10 to the container 12, and a medicament applicator 22 attached to a top side of the plate 18. The medicament applicator 22 is preferably replaceable and disposable.

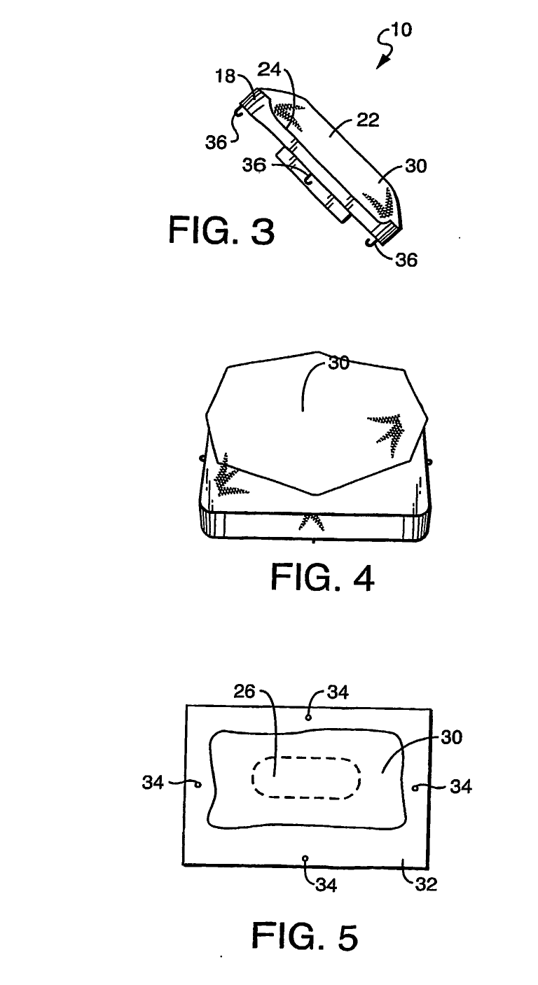

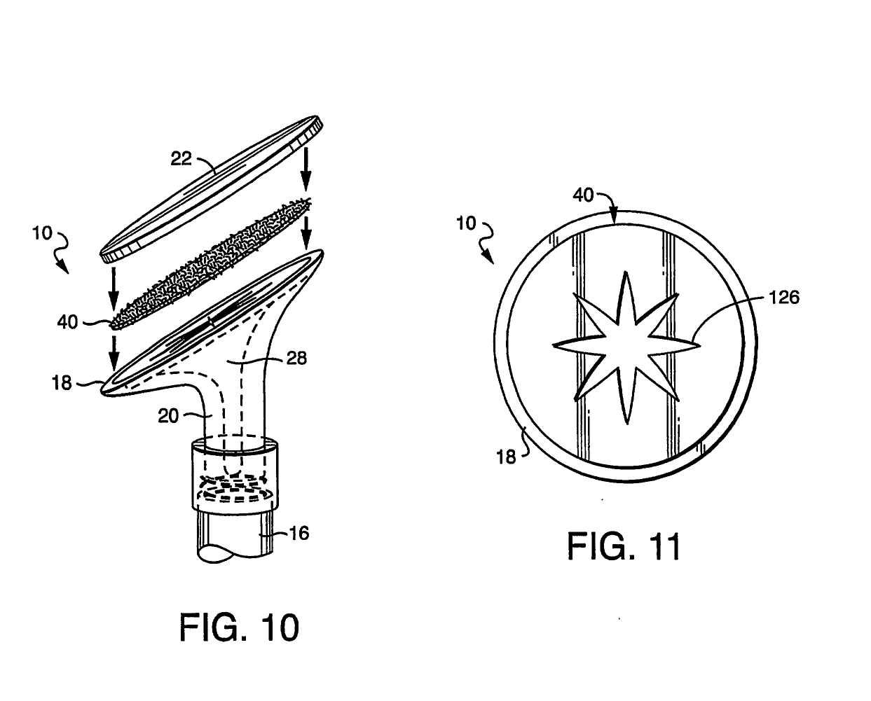

FIG. 3 is an enlarged side view of the medicament application device 10. As shown in FIG. 3, the plate 18 defines a central concave region 24 at the top side of the plate 18 for receiving the applicator 22. The plate 18 defines a hole 26 (as shown in FIGS. 5 and 7) at the central region of the plate 18, and the connector 20 defines an inner channel 28, which is in fluid communication with the hole 26. In use, the connector 20 is attached to the top of the stem 16 of the container 12, such that the inner channel 28 of the connector 20 is connected to the opening of the container 12, thus providing a continues passageway from the container 12 through the inner channel 28 and the hole 26 to the medicament applicator 22. The liquid medicament contained in the container 12 can pass through the passageway to the medicament applicator 22. Alternatively, as shown in FIG. 10, the inner channel 28 can have a relatively small inner diameter at the end coupled to the opening of the container 12 and have a relatively large inner diameter at the end coupled to the plate 18. The inner channel 28 may also be constructed with other diameter profiles, for example, with a uniform cross section along its length or with a decreasing cross section along its length.

The plate 18 may define at least one hole 26, as shown in FIG. 13, or one hole with a star-like shape, as shown in FIG. 11, to more effectively spread the liquid medicament to the applicator 22. The hole 26 and the inner channel 28 (including the diameter of the inner channel 28) may be constructed with various shapes and sizes according to the viscosity of the liquid and/or the desired application area or other application parameters.

FIGS. 1 and 2 show that the plate 18 has a rectangular shape. The plate 18 also can be manufactured with other shapes, for example, circular shape, oval shape, or other suitable shapes. The plate 18 is preferably made of a rigid material, e.g., plastic.

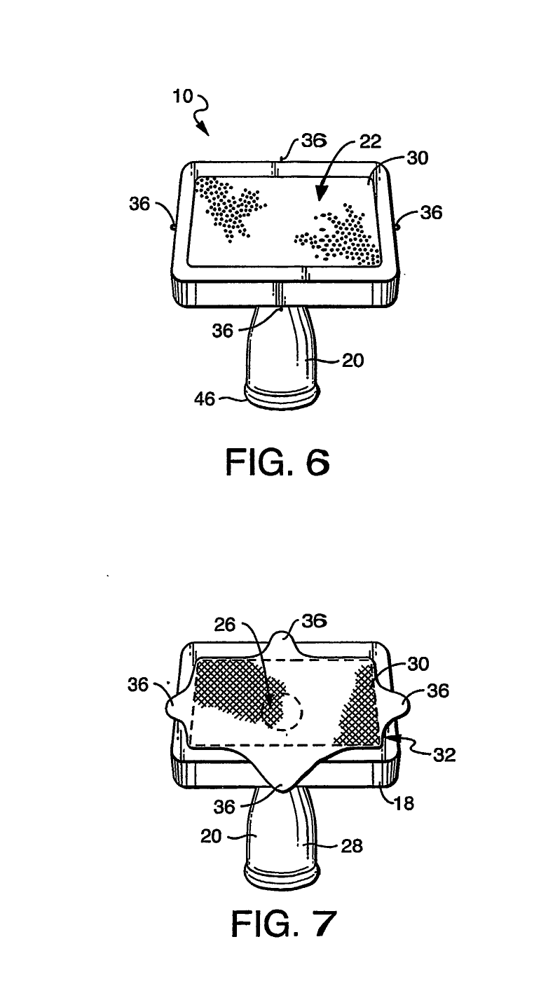

The medicament applicator 22 is preferably made from a liquid permeable or porous material that allows fluid to pass through, and at the same time, spreads the fluid to an application surface of the applicator 22. Exemplary materials include but are not limited to sponge, pledget, and cloth. In preferred embodiments shown in FIGS. 1-9, the medicament applicator 22 includes a sponge 30, which has a lower portion received in the central concave region 24 of the plate 18. In one preferred embodiment, the applicator 22 further includes a cover 32, which is preferably made from an elastic mesh or cloth, or other perforated materials, for securing the sponge 30 to the plate 18. In a preferred form, the cover 32 defines holes 34 at the peripheral edges of the cover 32, and the plate 18 includes hooks 36 extending from positions near the bottom peripheral edges of the plate 18. In use, as shown in FIGS. 6 and 7, the lower portion of the sponge 30 is received in the central concave region 24 of the plate 18, and is covered by the mesh 32, and the holes 34 on the mesh 32 are secured to the hooks 36 on the bottom side of the plate 18, so that the sponge 30 is secured on the plate 18 and is not movable during use. In the embodiments shown in FIGS. 1-9, the device 10 has a rectangular shape and includes four hooks 36 disposed near four bottom edges of the plate 18, and correspondingly, the cover 30 defines four holes near four edges of the cover 30, as shown in FIGS. 4 and 5, or at four corners, as shown in FIG. 7. The sponge 30 and mesh 32 can be removed from the plate 18 by detaching the mesh 32 from the hooks 36. The sponge 30 can be attached to the plate 18 by other suitable means, which preferably allow the sponge 30 to be removed and replaced without damaging the device 10.

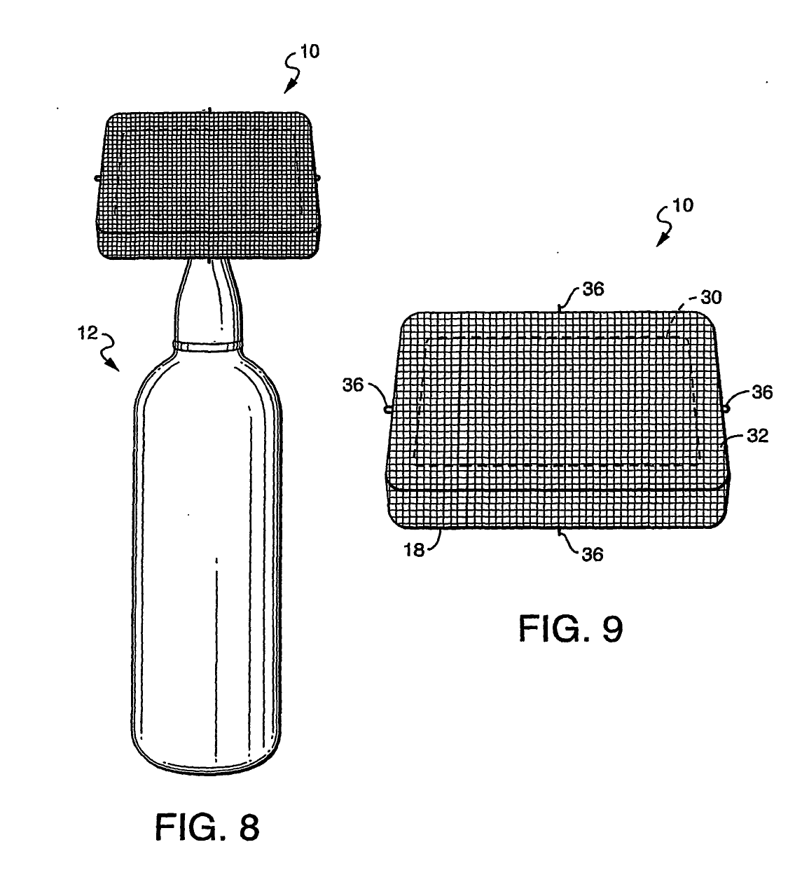

FIGS. 8 and 9 show an alternative embodiment which is similar to the embodiment shown in FIGS. 1-7. As shown in FIGS. 8 and 9, the device 10 is provided with a rectangular sponge 30, which is covered by a mesh netting 32. The mesh netting 32 is affixed to the plate 18 by the hooks 36, so that the sponge 30 is secured on the plate 18 and is prevented from moving during use.

FIG. 10 illustrates a side view and FIG. 11 illustrates a front view of a further embodiment of the present invention. Referring to FIG. 10, the plate 18 includes an array of resilient hooks 40, such as VELCRO® hooks, on a porous backing, extending from the top side of the plate 18. The medicament applicator 22 is a cloth or other porous material adapted to be attached to the hooks 40. The applicator 22 can be removed from the hooks 40 and can be replaced. The inner channel 28 extends through the connector 20, and preferably, has a relatively small diameter proximal end, which is connected to the opening of the container, and a relatively large diameter distal end which is connected to the bottom side of the plate 18. The relatively large diameter distal end preferably is provided with a trumpet-like shape, and the hole 26 defined in the plate 18 preferably has a star-like shape having multiple fingers extending outwardly, so that the liquid medicament passing through the inner channel 28 and the hole 26 can be distributed evenly across the bottom side of the cloth 22.

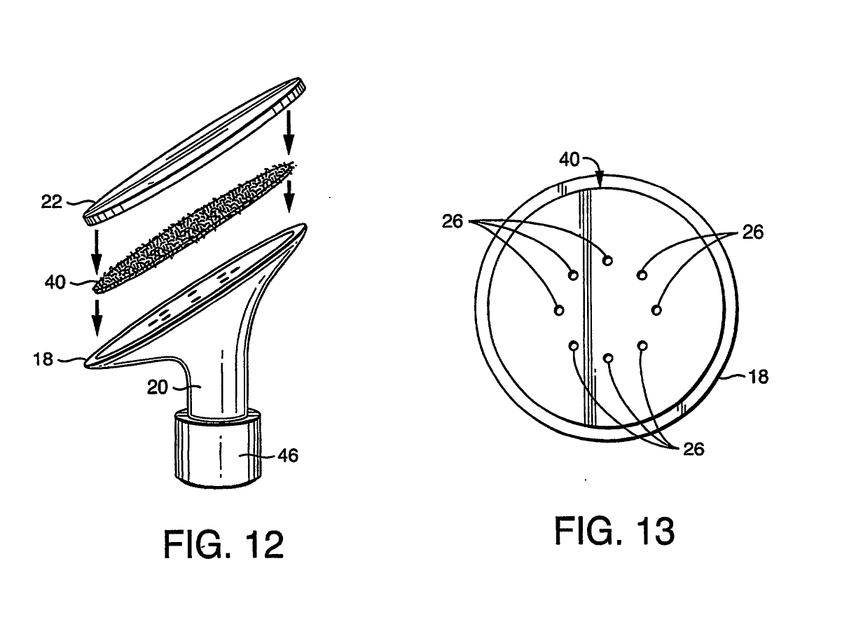

FIG. 12 shows a side view and FIG. 13 shows a front view of another preferred embodiment. Similar to the embodiment shown in FIGS. 10 and 11, the plate 18 of the device 1 0 includes an array of resilient hooks 40 on a porous backing extending from the top side of the plate 18. The medicament applicator 22 is a cloth adapted to be attached to the hooks 40. The applicator 22 can be removed from the hooks 40 and can be replaced. The plate 18 defines multiple holes 26 across the plate. The holes 26 are in fluid communication with the inner channel 28 of the connector 28. The liquid medicament passes from the container 12 through the inner channel 28 and the multiple holes 26 to the cloth 22.

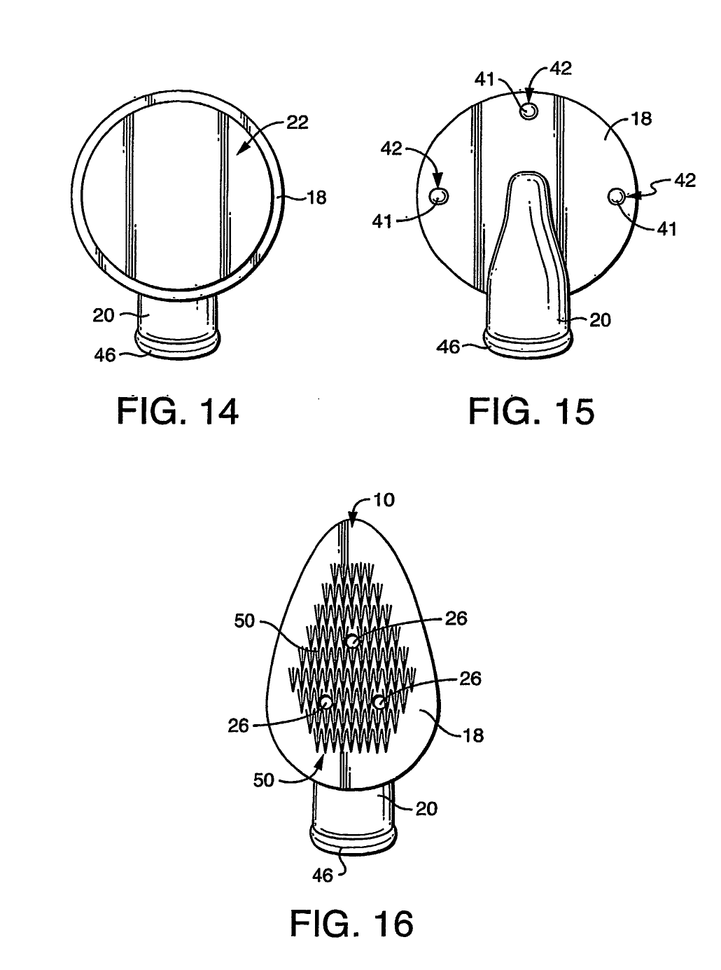

FIG. 14 is a front view and FIG. 15 is back view of a further alternative embodiment of the present invention. As shown in the figures, the device 10 includes a plate 18, a medicament applicator 22, and a connector 20 for connecting the device 10 to the container 12. The medicament applicator 22 preferably is a sponge and is replaceable and disposable. The applicator 22 includes relatively small protrusions 41 (for example, three protrusions) extending from the bottom side of the applicator 22 near the peripheral edge of the bottom side of the applicator 22. The small protrusions 41 can be pushed to snap-fit into associated relatively small holes 42 defined through the plate 18, so that the applicator 22 is secured on the plate 18. The applicator 22 can be removed from the plate 18 by pulling the applicator 22 in an opposite direction, or by pushing the protrusions 41 out of the holes 42 from the bottom side of the plate 18.

FIG. 16 shows a front view of another preferred embodiment of the present invention. As shown in FIG. 16, the device 10 includes a plate 18, a connector 20 extending from the bottom side of the plate 18 for connecting the device 10 to the container 12, and bristles 50 on a porous backing extending from the top side of the plate 18 for applying medicament to the skin of the mammal. The plate 18 defines multiple holes 26, for example, three holes as shown in FIG. 16, among the bristles 50. The holes 26 are connected to the inner channel 28 of the connector 20 for supplying medicament to the bristles 50 from the container 12. In use, the bristles 50 guide the application of the liquid medicament, which is supplied from the container 12 through the inner channel 28 and the holes 26, to the skin of the mammal.

The connector 20 of the device 10 preferably extends from the bottom side of the plate 18 to a proximal end 46, which preferably is detachably connected to the top end of the stem 16 of the container 12. As best shown in FIG. 2, the plate 18 and the connector 20 preferably form an angle and the applicator 22 of the device 10 is oblique relative to an axis X of the container bottle 12, so that a user can hold the container bottle 12 and can easily apply the applicator 22 to a skin surface. The proximal end 46 of the connector 20 preferably defines threads, as shown in FIGS. 14 and 15, adapted for engaging threads at the end of the stem 16, for connecting the connector 20 to the stem 16. Alternatively, the proximal end 46 can snap-fit onto the top end of the stem 16, as shown in FIG. 10. The connector 20 and the plate 18 are preferably constructed integrally. Alternatively, the connector 20 and the plate 18 can be connected by a pivot or a socket-ball connection, which defines a continuous inner channel therethrough and which allows relative rotatable movement between the plate 18 and the connector 20.

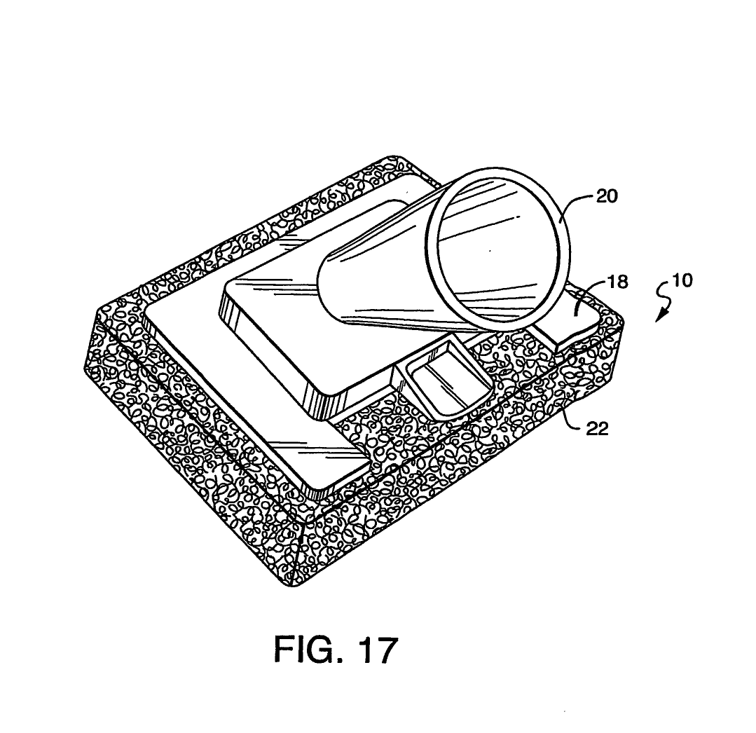

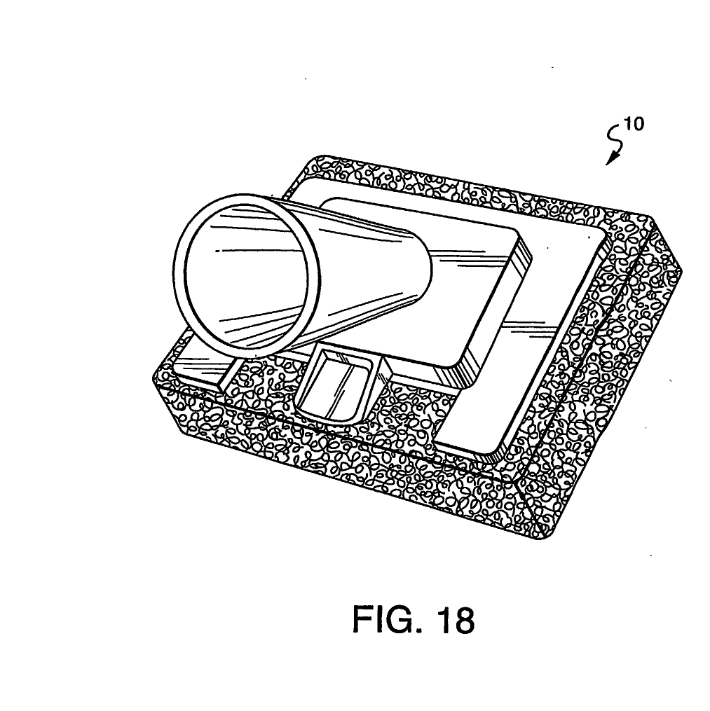

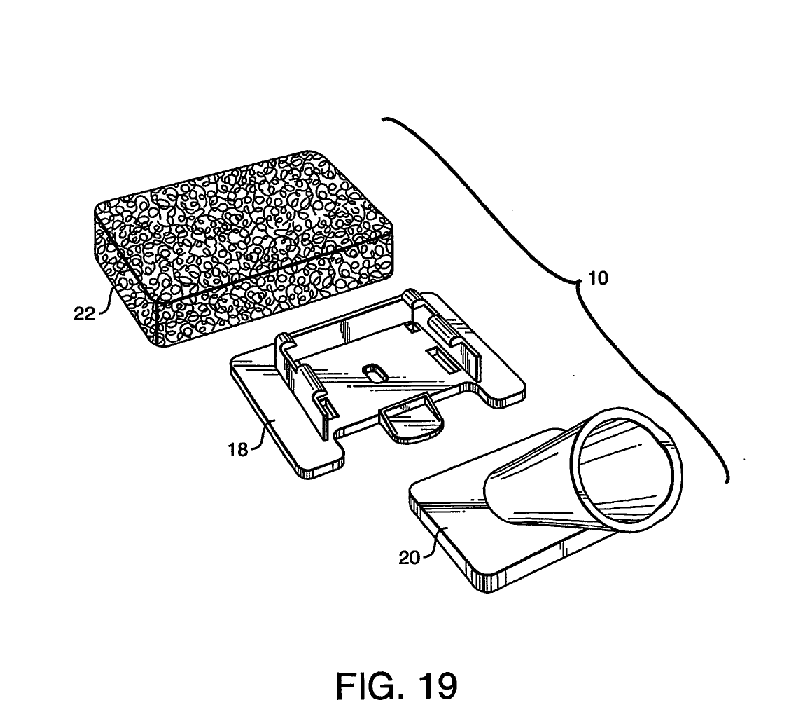

FIGS. 17-22 illustrate another preferred embodiment of the present invention. FIGS. 17 and 18 are oblique views of the medicament application device 10 viewed from different angles. FIG. 19 is an exploded view of the device 10. As best shown in FIG. 19, the device 10 includes a plate 18, a connector 20, and an applicator 22, which can be assembled to the device 10 shown in FIGS. 17 and 18.

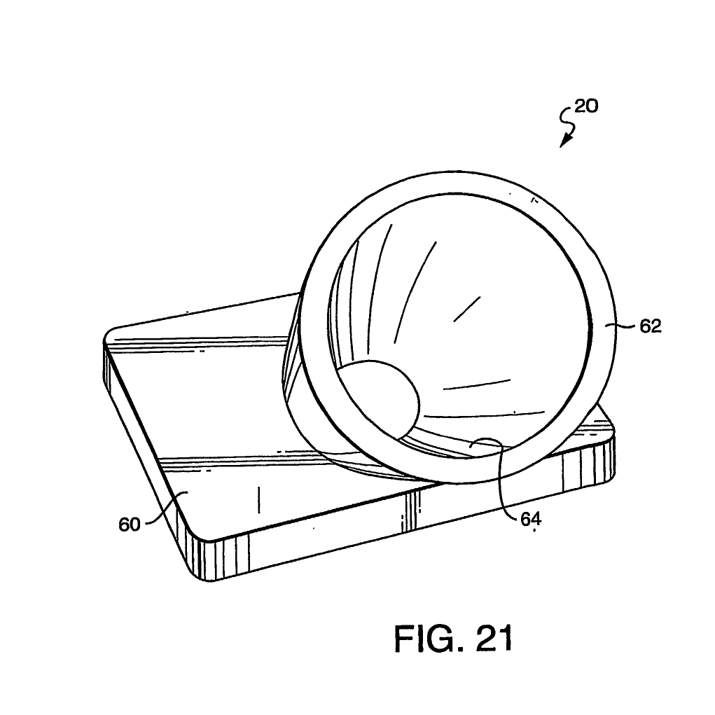

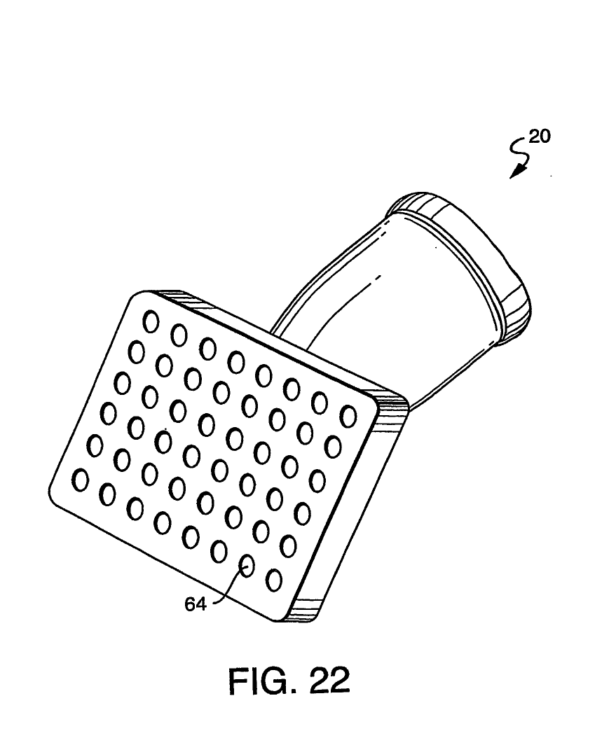

FIG. 21 is a back-oblique view and FIG. 22 is a front-oblique view of the connector 20. The connector 20 includes a plate portion 60 and a stem portion 62 extending from the back of the plate portion 60. The stem portion 62 defines an inner channel 64 therethrough for conducting medicament from the medicament container to the plate portion 60. In one preferred embodiment, threads are defined at the inner surface of the inner channel 64 to engage the threads of the stem 16 of the medicament container 12. The connector 20 may be connected to the medicament container 12 by other means, for example, snap-fitting. The plate portion 60 defines at least one, preferably a plurality of holes 66, as shown in FIG. 22. The holes 66 are in fluid communication with the inner channel 64 and are adapted to conduct the medicament uniformly to the plate 18.

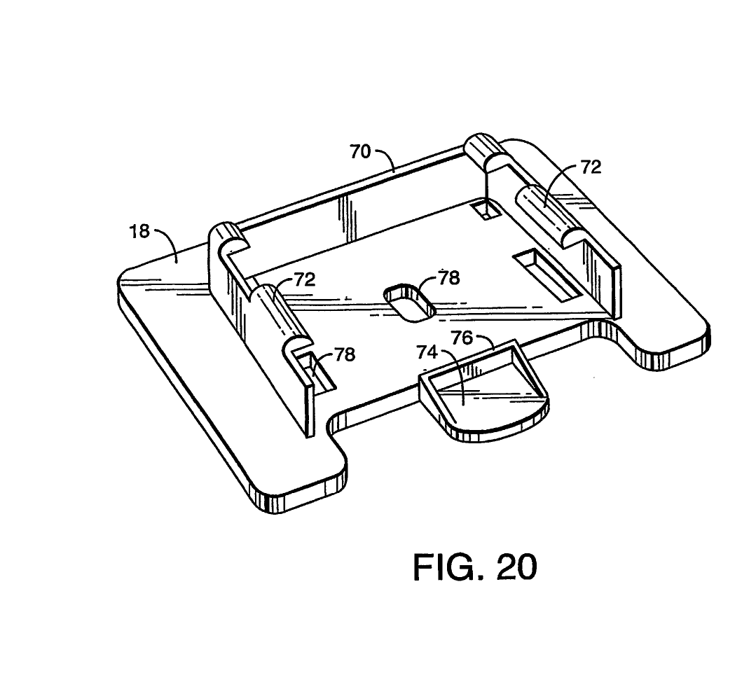

The plate 18, as shown in FIG. 20, in one preferred form, includes walls 70 extending upward from one side of the plate 18, and flanges 72 extending inward from the top of the walls 70. The walls 70 define an interior region with an opening at a back edge of the plate 18. The plate 18 further includes a tab 74, which is preferably resilient, extending from the back edge of the plate 18. A relatively small flange or protrusion 76 extends upward from the tab 74. The plate portion 60 of the connector 20 can be slid into the interior region defined by the walls 70 from the opening over the relatively small flange 76, and is locked in the interior region by the walls 70, flanges 72, and the relatively small flange 76. When a user presses the tab 74 downward, the plate portion 60 can be released from the opening and the connector 20 can be detached from the plate 18, so that a user can replace or clean the plate 18 and the applicator 22, or the connector 20. The connector 20 may be attached to the plate 18 by other suitable, preferably releasable means. The plate 18 defines at least one, preferably a plurality of holes 78 for conducting the medicament from the connector 20 to the applicator 22. The holes 66 in the plate portion 60 of the connector 20 and the holes 78 in the plate 18 are preferably defined in patterns that can optimally permit delivery of medicament to a bottom side of the applicator, preferably, in a spatially uniform manner.

The applicator 22 preferably is made from a flexible porous material, for example, sponge, for conducting and applying the liquid medicament uniformly to the skin of a mammal. As shown in FIGS. 17 and 18, the applicator 22 is preferably attached to the plate 18 by gluing or by other suitable means. As described above, the plate 18 and the applicator 22 can be detached from the connector 22 and can be disposed and replaced, thereby preventing transmission of skin diseases from one animal to another.

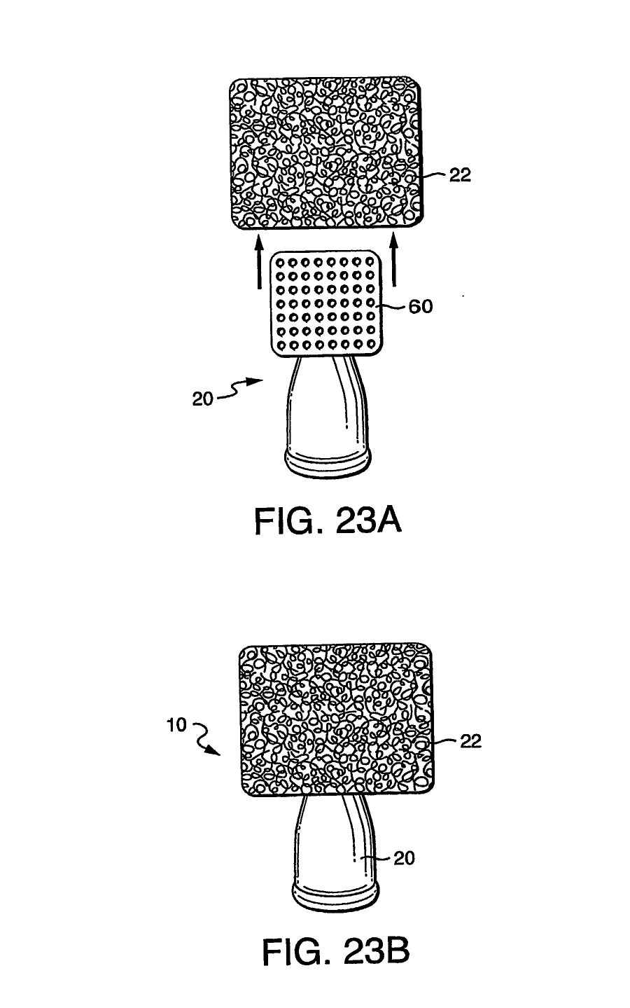

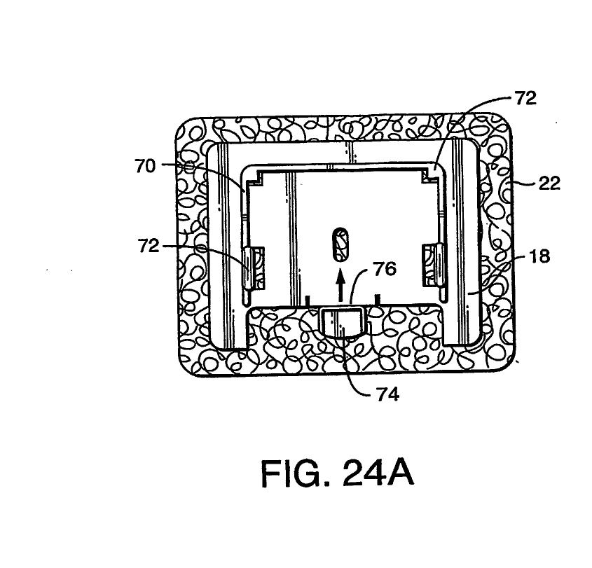

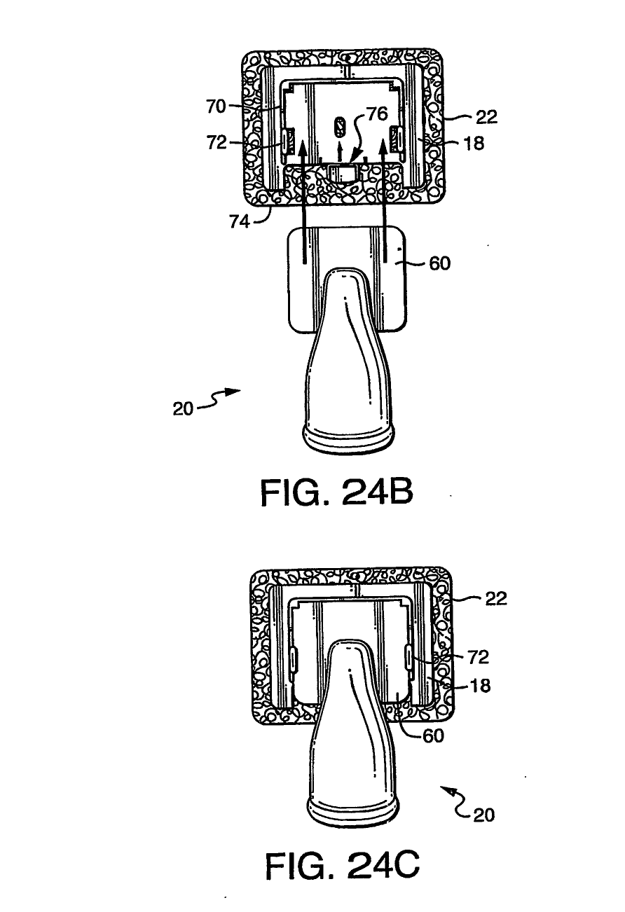

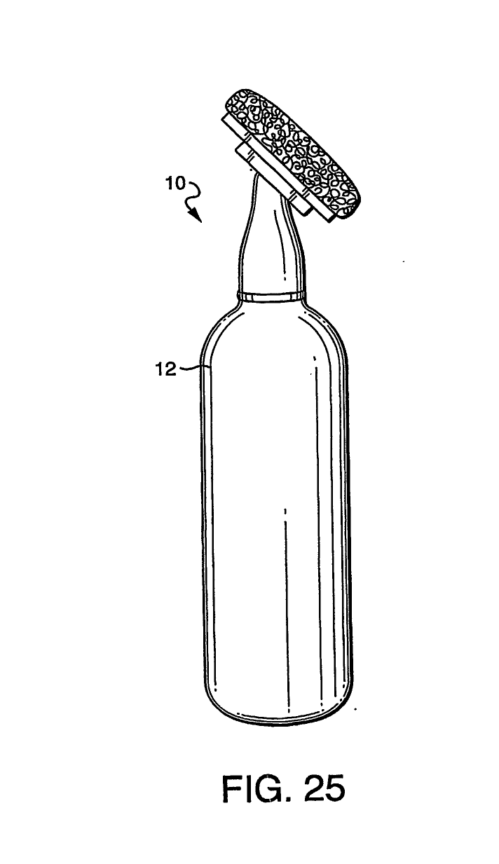

FIGS. 23A and 23B show front views and FIGS. 24A-24C show back views of how the medicament application device 10 is assembled. FIG. 24A shows a back view of the plate 18 with the applicator 22 attached to the front side of the plate 18. As shown in FIG. 23A (front view) and FIG. 24B (back view), the plate portion 60 of the connector 20 is slid into the interior region defined at the back side of the plate 18, and is locked in the interior region by the walls 70, flanges 72, and the relatively small flange 76 on the tab 74, as shown in the finished views in FIG. 23B (front view) and FIG. 24C (back view). FIG. 25 shows that the medicament application device 10 is connected to the medicament container 12.

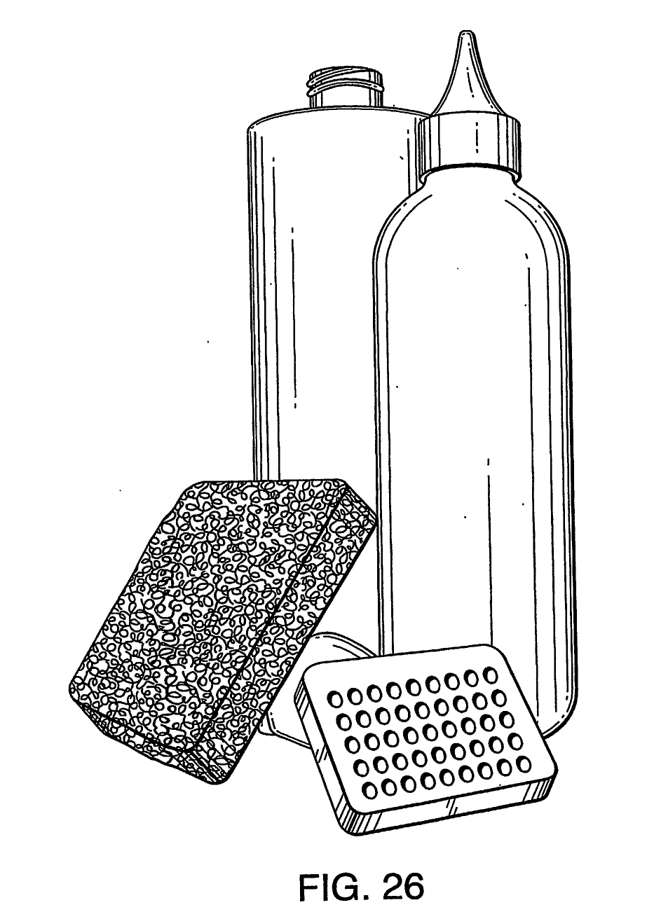

FIG. 26 illustrates an exemplary embodiment of a rinse applicator kit according to one aspect of the present invention. The rinse applicator kit is designed to allow precise dilution of the rinse concentrate and easy application of the diluted rinse solution on the animal. The disposable sponges are intended to be used once and then discarded. Depending on the type of skin infection, the veterinarian may recommend that a sponge be used on only a single animal, even if more than one animal has the infection in the same environment.

The rinse applicator kit preferably includes:

(i). Rinse—8 fl. oz. concentrate;

(ii). Measuring Cup—1 fl. oz.—(preferably multiple units, e.g. 3 units);

(iii). Applicator Bottle—16 fl. oz.;

(iv). Applicator Attachment;

(v). Disposable Sponges—(preferably multiple units, e.g. 16 units).

The rinse preferably includes active ingredients: Miconazole base 5.2% w/w, Chlorhexidine Gluconate 5.9% w/w. After dilution, the ingredients of the rinse are equivalent to 0.2% Miconazole Nitrate and 0.2% Chlorhexidine Gluconate. The rinse provides antibacterial and antifungal agents for optimal therapeutic effectiveness. The rinse is preferably used for dermatological conditions associated with infections responsive to Miconazole Nitrate and Chlorhexidine Gluconate.

Before first use of the rinse applicator kit, the operator first unscrews the cap, removes the protective liner of the concentrate container, then dilutes the concentrate at a rate of ½ fluid ounce of the concentrate to 16 ounces of water, and applies the diluted solution liberally to the animal's hair coat, paying particular attention to infected areas. The diluted solution should be used within eight hours and the remaining solution should be discarded. It is preferred to allow the solution to remain on the animal over an effective amount of time. The operator should not allow the animal to lick the treated areas until dry to prevent ingestion. The medicament should be applied twice weekly until symptoms subside, then weekly, or as directed by the veterinarian. The concentrate is preferably stored upright at room temperature.

For topical use on dogs, cats, or horses, the operator should avoid any contact of the rinse with eyes and niucous membranes of the animals. If eye contact or skin irritation develops, the operator should rinse the eyes or the skin of the animal thoroughly with water, discontinue treatment, and immediately consult a veterinarian.

The following (1)-(8) items are step-by-step instructions of how to use the rinse and the rinse applicator kit:

(1). Read and understand the entire rinse concentrate label before use of the product with the rinse applicator kit.

(2). Attach a disposable sponge to the applicator attachment by sliding the rectangular applicator head into the plastic rectangular receptacle on the back of the sponge until the pieces snap securely in place.

(3). Open the cap on the rinse concentrate container, remove the foil liner from the container, and carefully pour the concentrate into the measuring cup up to the ½ fl. oz. line. Pour from the concentrate from the measuring cup into the 16 fl. oz. applicator bottle. Close the cap on the rinse concentrate container.

(4). Fill the applicator bottle with 16 oz. of water. Secure the applicator attachment with the attached sponge onto the applicator bottle until snug. Do not over tighten. Use the solution within 8 hours of dilution and discard any remaining solution after this time.

(5). Invert the bottle several times over a sink or tub to mix the concentrate and water. Invert the bottle and squeeze gently to saturate the sponge with the treatment solution. (Gently pressing the surface of the sponge onto the hard surface of the sink or tub will facilitate more rapid moistening of the entire sponge.)

(6). Treat the areas of the animal's hair and skin until wet as directed by a veterinarian. The bottle should be kept inverted for proper delivery of the solution to the sponge. The amount of solution delivered to the sponge can be controlled by how much the bottle is squeezed. Dab the sponge onto affected areas for local treatment Use gentle strokes of the sponge as if petting the animal to treat larger areas of the body. Do not rinse. Do not allow animal to lick the treated areas until dry to prevent ingestion.

(7). When finished, remove the applicator attachment from the applicator bottle. Press the tab on the back of the sponge to slide it off the applicator attachment head. Discard the sponge and any remaining solution in the applicator bottle.

(8). Wash the measuring cup, applicator attachment, and applicator bottle several times with warm water and allow to dry until next use.

The invention may be embodied in other specific forms without departing from the spirit or essential characteristics thereof. The present embodiments are therefore to be considered in all respects illustrative and not restrictive, the scope of the invention being indicated by the appended claims rather than by the foregoing description, and all changes which come within the meaning and range of equivalency of the claims are therefore intended to be embraced therein.

What is claimed is:

1. A medicament application device adapted for using with a medicament container, said medicament container having an opening for dispensing medicament contained in said container to the device, said medicament application device comprising:
a plate defining at least one hole therethrough;
a connector extending from a bottom side of said plate to a proximal end, said connector proximal end being adapted for connecting to said opening of said container, wherein said connector defines an inner channel therethrough, said inner channel being in fluid communication with said opening of said container and said hole of said plate; and
a medicament applicator detachably attached to a top side of said plate, said medicament applicator being made from a fluid permeable material.

2. A medicament application device according to claim 1, wherein said medicament applicator is made from sponge.

3. A medicament application device according to claim 1 further comprising a cover disposed on a top surface of said medicament applicator, said cover being detachably attached to said plate to secure said medicament applicator on said plate, wherein said cover is made of a fluid permeable material.

4. A medicament application device according to claim 3, wherein said cover is made from a mesh.

5. A medicament application device according to claim 1, wherein said plate comprises a plurality of flexible hooks extending from the top side of the plate, and said applicator is detachably attached to said plurality of flexible hooks.

6. A medicament application device according to claim 1, wherein said plate defines a plurality of relatively small holes near peripheral edges of the plate, and said medicament applicator includes a plurality of protrusions extending from a bottom surface of said medicament applicator, said protrusions being adapted to snap-fit into said relatively small holes.

7. A medicament application device according to claim 1, wherein said at least one hole comprises a plurality of holes.

8. A medicament application device according to claim 1, wherein said at least one hole has a star-like shape.

9. A medicament application device according to claim 1, wherein said applicator includes a porous backing and bristles extending from said porous backing, said porous backing being detachably attached to the top side of said plate.

10. A kit including a medicament application device according to claim 1, and further comprising:
a medicament container for connection with the connector.

11. A kit according to claim 10, wherein said kit further includes a bottle of solution.

12. A kit according to claim 11, wherein said bottle of solution contains a medicament selected from the group comprising:
(a) moisturizing bath rinses;
(b) non-steroidal and steroidal anti-inflammatories and/or anti-pruritics;
(c) anti-infectives; and
(d) anti-parasitics.

13. A kit including a medicament application device according to claim 1, and further comprising:
a plurality of the medicament applicators for detachable attachment to a top side of the plate.

14. A medicament application device adapted for using with a medicament container, said medicament container having an opening for dispensing medicament contained in said container to the device, said medicament application device comprising:
a plate defining at least one hole through said plate;
a connector extending from a bottom side of said plate to a proximal end, said connector proximal end being adapted for connecting to said opening of said container, wherein said connector defines an inner channel therethrough, said inner channel being in fluid communication with said opening of said container and said hole of said plate; and
bristles extending from a top side of said plate.
